(19) United States
(12) Patent Application Publication
(10) Pub. No.: US 2004/0185066 A1
(43) Pub. Date: Sep. 23, 2004

(54) Antifreeze gel in a deformable container

(76) Inventor: Yuh-Jye Uang, Chesterfield, MO (US)

Correspondence Address: Paul M. Denk, Suite 170, 763 S. New Ballas Road, St. Louis, MO, 63141, US

(21) Appl. No.: 10/797,947

(22) Filed: Mar. 11, 2004

Related U.S. Application Data
(60) Provisional application No. 60/454,592, filed on Mar. 17, 2003.

(51) Int. Cl. A61L 9/04; A61K 9/00

(52) U.S. Cl. 424/400

(57) ABSTRACT

An antifreeze gel composition is provided which particularly useful to prevent ice formation. The composition includes a water-soluble antifreeze agent such as sorbitol, dextrose; gel forming agent; catalysts; and additives. The present invention further provides a rigid, good transparent gel. The advantage of the hard gel is to provide multi-color printing images on the surface of the deformable container. The method of printing process can be either screen printed or pad printing. The hard gel was formed by two-part reaction between gum and cross-linking agents. The hardness of the container is below 30 A and the elongation of the container is over 1100%. The container is soft, stretchable, and it is a closed system.

### CROSS REFERENCE TO RELATED APPLICATION

This non provisional patent application claims priority to the provisional patent application having Ser. No. 60/454,592, having a filing date of Mar. 17, 2003.

### TECHNICAL FIELD OF THE INVENTION

The invention relates generally to clear antifreeze gel composition. More particularly, the present invention relate of clear antifreeze gel composition useful to preventing the ice formation in a containers. The gel is rigid and transparent. Since the gel is rigid in the container, multi-color images can be printed on the surface of the container. The different shapes of cross-linked polymer gel can be suspended in the gel within the container. After squeeze, the rigid gel will become into tiny gels and shapes of cross-linked polymer gel will be flowed around. The containers is preferably shaped or decorated according to the theme. Moreover, the materials of the container can be transparent, soft and stretchable plastics. The hardness of the container is below 30 A and the elongation is larger than 1100%.

### BACKGROUND OF THE INVENTION

In general, the clear or transparent toys containing gel for amusement, creative play, and medical stress release products are particularly attractive because consumer likes transparency to quality. In recent years, there are more and more commercial products which its design have gel filled in a closed container such as glass, acrylic, polycarbonate, polyvinyl chloride and thermoplastic rubber and the like in gifts, toys, and medical industry. The complication associated with the transportation and storage of this aqueous gel filled products, which can be frozen during the winter, has became a major commercial problem. Most of aqueous gel containing products in the market is either having no or very little antifreeze effect. Since the gel inside the container is too soft, it is very difficult to print the multi-color images on the surface of the container by conventional printing methods. Some investigation has been approached to make gel that has antifreeze effect. U.S. Pat. No. 4,315,779 to Heyd et,al. disclosed a gel comprises about 1 to about 10% by weight of a hydrophilic cellulose polymer, an alginate or xanthan gum, from about 5 to about 40% by weight of glycerin, sorbitol propylene glycol and from about 50 to about 95% by weight water. U.S. Pat. No. 4,439,337 to Nimerick et al. disclosed antifreeze gel comprising 1) about 5.0 to about 95.0% polyhydroxy compounds such as ethylene glycol, propylene glycol, 2) about 0.1 to about 45.0% salt which function to reduce the freezing point such as CaCl₂. sodium acetate. 3) about 1 to about 10% of organic polymer such as cellulose polymer, polyacrylamide, plyacrylic acid polymer. There are some aircraft anti-icing fluids, thickened by gel forming gums, was formed in a very high amount of propylene glycol or ethylene glycol.

There are some aircraft anti-icing fluids, thickened by gel forming gums, was formed in a very high amount of propylene glycol, ethylene glycol or salt. U.S. Pat. No. 4,698,172 to Tye et al. disclosed anti-icing gel comprising a glycol-based aqueous solution thickened essentially with gel-forming carrageenan, in amount of less than 5 weight percent. U.S. Pat. No. 5,385,688 to Miller et al. disclosed the composition of antifreeze gel consist of alkylene glycol, polymeric thickeners, and water. The polymeric thickener is selected from the group consisting of polyacid thickener, gums, starches, modified starch, and cellulose. U.S. Pat. No. 5,591,375 to Loft et al. disclosed antifreeze composition comprising two parts, part A and part B. Part A contains about 0.1 to 20% by weight of salt of alginic acid, about 20 to 99% by weight water and between about 0.1 to about 50% propylene glycol. Part B contains about 0.006 to 15% by weight of polyvalent cation about 20 to 99% by weight water and between about 0.1 to about 50% propylene glycol.

### SUMMARY OF THE INVENTION

The present invention provides transparent antifreeze gel composition containing water, antifreeze agent, gel forming agent, and inorganic salts, optionally contains different shapes of polymer gel. The hard gel was formed in two part reaction, the part A contains gum, antifreeze agent, water and the compositions of part B are citric acid, buffer solution, and inorganic salts functioned as catalyst. In use, part A solution was dissolved by heat and filled into container first then followed injected by part B. The materials of the container include thermoplastic rubber, silicon rubber, and the like. The cap sealed container can be deformed and stretchable. Moreover, a polysaccharide forms a flexible, translucent cross-linked polymer gel in different shapes through the reaction with multivalent cations. The cross-linked polymer gel can be suspended inside the antifreeze gel. Further, cross-linked polymer gel can be colored by dyes, or pigments, holographic powders and can have other interesting colors or effects, such as glowing in the dark, changing colors with temperature, and so on.

It is an object of the present invention to provides the formulation by varied the components in any number of different ways to produce the gel antifreeze product.

It is another object of the present invention to provide antifreeze gel composition is non-toxic, non-sensitizing, no harmful vapor emissions, no flash point and dried residue is nonflammable.

It is still another object of the present invention to provide multi-color images printing on the surface of a very soft, stretchable container which containing rigid gel.

Further objects and advantages of the subject invention will be apparent to those skilled in the art.

### DETAILED DESCRIPTION OF THE INVENTION

In the present invention, antifreeze gel composition comprising water, antifreeze agent, gel forming agent, and inorganic salts. In use the rigid gel was formed by two part reactions. The surface of the container, which contains the hard gel, can be printed with multi-color images.

Antifreeze Agent

The sweeteners used as antifreeze agents in the present invention include sugar alcohols, corn syrup, maltodextrin, sucrose, lactose, maltose and dextrose. Sugar alcohols, or polyols, are chemically alcohols, but are derived from sugar molecules. They include erythritol, hydrogenated starch hydrolysates, isomalt, lactitol, maltitol, mannitol, sorbitol, and xylitol. The mannitol, sorbitol, and xylitol are monosaccharide polyols while the isomelt, lactitol and maltitol are disaccharide polyols. Depend on the different of D.E. (dextrose equivalent); the effect of the freezing depression point is also varied. Both sucrose and starch have zero value of D.E. Maltodextrin product with D.E. values is from 6-22. The corn syrup itself is divided into four types of corn syrups on the basis dextrose equivalent (D.E.). The type I is from 20 to 38 D.E., type 11 is from 38 to 58 D.E., type III is from 58 to 73 D.E., and type IV is from 73 D.E. and above. The D.E. value of dextrose is 100. The freezing point depression, lower water activity and inhibition of water crystallization will be increased with increasing DE. The most useful sweeteners compounds include dextrose, corn syrup and sugar alcohol can be utilized in the invention. These three ingredients can help prevent ice crystallization. The dextrose is consisting of low molecular weight polymer of glucose. Dextrose, monosaccharides that is smaller molecule, lowers the freezing point more than disaccharides such as sucrose, which its DE is, zero. Also the degree to which the freezing point depressed is function of the amount of dissolved molecules in the solution. The freezing point of 20% of dextrose is −5° C. and at 40% of dextrose, its freezing point is −14° C.

Sugar alcohols are sweeteners among commonly added to depress the freezing point to various degrees. The smaller the molecular weight of the sugar alcohol, the greater the freezing point depression because it is able to dissolve more the smaller molecule and bind up the water. Preferred the sugar alcohols are monosaccharide polyols such as mannitol, sorbitol and xylitol. Mixture of the sweeteners may be employed. Preferred are compositions employing only the sugar alcohol. The sugar alcohol and/or corn syrup, dextrose of the antifreeze agent is present in amounts ranging from about 20 to about 80 weight percent based on the total weight of the antifreeze agent components, preferably from about 20 to about 60%, and more preferably about 40 to about 55%.

Gel Forming Agent

In the present invention, using gums and/or the mixtures made Gel. Gums can be classified according to their structure or function, but are most often grouped together by their sources. Most are considered natural products, depending on how they become the final form. The most widely used of the plant exudates is gum Arabic, but gum tragacanth, gum karaya and gum ghatti, so fall into this category. Others are considered plant extracts, such as konjac and pectin. Guar and locust bean gum are seed gums while the seaweed extracts include agar-agar, alginate, and carrageenan. Microbial polysaccharides produced as microbial exudates include xanthan gum, curdlan and gellan gum. Cellulose such as microcrystalline cellulose, methyl cellulose and hydroxypropyl methyl cellulose are modified natural products. In addition, some products are derived from animal sources that because of their functionality are often considered gums, such as gelatin, and chitosan. Under the right set of conditions, some gums do not merely thicken, but cross-link or otherwise join molecular using various types bond at junction zone to form three-dimensional networks called a gel. Most gel is thermally reversible and some are thermally irreversible. The strength of the gel depends on its structure and concentration, as well as ionic strength, PH and temperature. Various kind of gums used in the present invention including agar, alginates, carrageenan, cellulose gums, curdlan, chitosan, furcellaran, gelatin, gellan gum, guar gum, gum arabic, gum karaya, gum ghatti, gum tragacanth, konjac, pectin, pullulan, tara gum, locust bean gum, xanthan gum and the mixtures: In general, combining gums has very good advantage, synergistic reaction between or more gums. The xanthan gum interacts synergistically with tara, locust bean gum and konjac. It forms very strong and elastic gels on cooling when mixed with locust bean gum, which at 50:50 ratio. Mixtures of gums often exhibit nonadditive properties where sum functionality is greater than expected.

The preferred gum used in the invention is combination of locust bean gum and carrageenan gum. Carrageenan gum has three forms are known as iota, kappa and lambda. The iota and kappa carrageenan are preferred, and the kappa carrageenan is most preferred. The amount of gums in the aqueous liquid should be less than 5% weight, based on the water. The amount of gum is preferably within the range of about 0.05 to 3 weight %, and more preferably within the range from about 0.15 to about 2.0 weight %.

Catalyst

The antifreeze gel composition of the invention also includes at least catalyst. The catalyst, cross-linking agent, for gums in the invention includes alkali metal salts and alkaline earth metal salts such as, calcium chloride, magnesium chloride, sodium chloride, lithium chloride and potassium chloride. Preferred is potassium chloride. The Kappa and Iota carrageen have a strong interaction with potassium and calcium ion respectively. The presence of these cations increases the hardness, fragility, temperature of gelation, and the syneresis of other gels in water.

To make hard gel, the salts may be used in an amount of up to about 6% by weight of the gel material. Preferably, at least about 0.01% by weight of the salt is included, based on the total weight of the gel material. In a preferred embodiment, the salt is included in an amount of from about 0.2% to about 6% by weight, more preferably from about 0.05% to about 3% by weight, still more preferably from 0.1% to 1.0% based on the weight of gel.

Cross-Linked Polymer Gel

The antifreeze gel material can include other ingredients such as cross linked polymer gels. Preferred the gel was made from polysaccharide such as sodium alginate and or the mixture. In solution alginates can be used as thickening or gelling agent. These properties depend on the presence or absence of calcium ions. As a thickening agent, the process is heat reversible while as gelling agent, the alginate gel will be thermally irreversible. In this invention the calcium-induced gelation alginate is the most preferred. The cross-linked gel polymer is preferably shaped or decorated according to the theme. A coloring agent that provides special color or visual effects may be included into this alginate gel. Coloring agents are preferred as nontoxic pigments. Examples of colorants providing special color or visual effects include temperature-dependent colorants, glow in the dark pigment and the glitter flakes.

Other Components

The composition of this invention is formulated using a major proportion of water in addition to the antifreeze agent, gel-forming agent, and catalysts. The water used is preferably deionized before it is formulated in the composition. The amount of water comprise from about 20 to 90 weight % of the composition, preferably about 20 to 80 weight %, more preferably from about 40 to 60 weight %.

The antifreeze gel material can include other ingredients such as coloring agents. Most preferred as coloring agents are nontoxic dyes such as food dye. Other additives such as sodium phosphate, sodium citrate, potassium citrate and citric acid are to buffer the gel system to a relatively high PH, where carrageenan is more stable. Preservatives are also included sodium benzoate, potassium benzoate.

Container

The containers are preferably made of a plastic, and more preferably of a soft and flexible plastic. Moreover the container is preferably deformable and stretchable. In a preferred embodiment, the containers are composed of a thermoplastic rubber (TPR) compounds which have the hardness below 10 A.

Commercially available thermoplastic rubber type polymers which are specially useful in forming the compositions of the present invention are sold under trademark Kraton by Kraton Chemical company and trademark Septon by Septon Company of America. The most common structure is the linear ABA block type; styrene-butadiene-styrene (SBS) and styrene-isoprene-styrene (SIS) which is the Kraton D rubber series. Kraton G is another type of polymer. The copolymer comprises a styrene-ethylene-butylene-styrene (S-EB-S) structure. The Kraton G series is preferred in the practice of the invention. The optionally blended diblock polymers include the AB type such as styrene-ethylenepropylene(S-EP) and styrene-ethylenebutylene(S-EB), styrene-butadiene (SB) and styrene-isoprene (SI). Septon resins are available in either diblock(A-B) type the more common tiblock (A-B-A) types. These include a hydrogenated polyisoprene (S-EP, S-EP-S), a hydrogenated poly-isoprene/butadiene (S-EEP-S) polymer or a hydrogenated poly-butadiene (SEBS) polymer. Depend on the hardness of the thermoplastic rubber, preferred are compositions employing the combination of triblock and radical block of thermoplastic rubber. A preferred mineral oil to mix with thermoplastic rubber of the invention is so-called “white” mineral oil, which is water-white (i.e., colorless and transparent) and is generally recognized as safe for contact with human skin. Mineral oil may also be characterized in terms of its viscosity, where light mineral oil is relatively less viscous than heavy mineral oil. Any mineral oil may be used in the invention. The mineral oil can be light mineral oil or heavy mineral oil. Light mineral oils are preferred for use in the invention. The containers have the shapes may be flat or three-dimensional. The containers may be of different colors and may have special color effects, such as glow in the dark. The container may be scented and may also be formed either opaque or transparent. The container is preferably shaped or decorated according to a theme.

The invention is illustrated by the following example. The example is merely illustrative and does not in any way limit the scope of the invention as described and claimed. All parts are parts by weight unless otherwise noted.

### EXAMPLE 1

A transparent antifreeze gel is formed by two-part reaction. The part A includes 1.0 part by weight of carrageenan gum, 0.5 part by weight of locust bean gum, 0.005 part by weight of sodium benzoate, 40 part by weight of sorbitol, and 50 parts by weight of water. The part B includes 0.2 part by weight of potassium citrate, 0.3 part by weight of citric acid, 0.2 part by weight of calcium chloride; and 10 part by weight: The ingredients in Part B is dissolved into clear solution at room temperature while the ingredients in part A need to be heated at 75° C. to get clear solution. A hard, brittle, and transparent gel was formed by pouring the part B solution into part A solution.

### EXAMPLE 2

Add 2 g of sodium alginate to 100 ml of deionized water. Stir the suspension until it becomes homogeneous. The mixture was droped into a solution of 1 g of calcium chloride in 100 ml of deionized water. A flexible gel beads will form instantly as the sodium ions are exchanged with calcium ions and the polymer becomes cross-linked.

A transparent antifreeze-gel-in a deformable container can be prepared according to the Invention. A transparent thermoplastic rubber container, which has a shaped, likes a ball, which have circle hole on the bottom. Cross-linked polymer gel, shapes according to theme, can be put inside the ball and sealed the ball with the cap. The transparent antifreeze gel of Example 1 was formed by two-part reaction, part A was injected in the ball through the cap then part B solution was injected with the same procedure. After the hard gel was formed, multi-color images can be printed on the substrate of the container by either screen printed or pad printing machine.

1. A deformable container with an antifreeze gel comprising
Water
Antifreeze agent
Gel forming agent
Catalyst
Cross-linked polymer gel
Container.

2. The antifreeze agent of claim 1 and including at least one sweetener.

3. The antifreeze agent of claim 2 wherein said sweetener comprises the sugar alcohols consisting of erythritol, hydrogenated starch hydrolysates, isomelt, lactitol, maltitol, mannitol, sorbitol, and xylitol.

4. The antifreeze agent of claim 2 wherein said sweetener comprises corn syrup consisting of four types D.E. grade. The type I is from 20 to 38 D.E., type II is from 38 to 58 D.E., type III is from 58 to 73 D.E., and type IV is from 73 D.E. and above.

5. The antifreeze agent of claim 2 wherein said sweetener comprises dextrose that D.E values is 100.

6. The antifreeze agent of claim 2 wherein said sweetener comprises between from about 20 to about 80% by weight of gel.

7. The gel forming agent of claim 1 and including gums, said gums comprising agar, alginates, carageenan, cellulose gums, curdlan, chitosan, furcellaran, gelatin, gellan gum, guar gum, gum arabic, gum karaya, gum ghatti, gum tragacanth, konjac, pectin, pullulan, tara gum, locust bean gum, xanthan gum and mixtures thereof.

8. The gel forming agent of claim 7 wherein the gums preferably include from about 0.05 to about 3.0 percent by weight of gel.

9. The composition of claim 1 further comprises one or more catalysts.

10. The composition of claim 9 wherein the catalyst comprise from about 0.05 to about 2.0 percent by weight of gel.

11. The composition of claim 10 wherein the catalyst is selected from the group consisting of alkali metal salts and alkaline earth metal salts.

12. The composition of claim 11, wherein said the catalyst comprises potassium chloride.

13. The composition of claim 1 wherein the cross-linked polymer gel was made by reaction between seaweed extracts and alkaline earth salt.

14. The composition of claim 13 wherein said seaweed extracts comprise sodium alginate, propylene glycol alginate.

15. The composition of claim 13 wherein said seaweed extracts comprise sodium alginate.

16. The composition of claim 13 wherein said the alkaline earth salt is selected from the group consisting of calcium chloride, magnesium chloride.

17. The composition of claim 13 wherein said the alkaline earth salt comprise calcium chloride.

18. The composition of claim 1 wherein further comprises buffer agents.

19. The composition of claim 18 wherein buffer agents are selected from the group consisting of potassium phosphate, potassium citrate, sodium citrate, and citric acid.

20. The composition of claim 1 wherein further comprising a colorant.

21. The composition of claim 1 wherein further comprising preservatives.

22. The composition of claim 21 wherein the preservative is sodium benzoate.

23. The container composition of claim 1 wherein said the mixture between thermoplastic rubber with mineral oil.

24. The container composition of claim 1 wherein said the hardness is below 30 A.

25. The container composition of claim 1 wherein said the elongation is larger than 1100%.

26. A deformable container, comprising a transparent gel and varied shapes of cross-linked polymer gel are suspended inside of antifreeze gel.

27. The cross-linked polymer gel composition of claim 24 wherein further comprising a colorant.

28. The cross-linked polymer-gel composition of claim 24 wherein further comprising glow in the dark pigment.

29. The cross-linked polymer gel composition of claim 24 wherein further comprising a material that provides an effect selected from the group consisting of fluorescence, pearlescence, metallic appearance, glitters, and sparkle.

30. A container according to claim 1 that the substrate can be printed with multi-color, images by either screen printed or pad printing machine.

31. A transparent antifreeze gel comprising
about 20-80 by weight of antifreeze agent
about 0.05-3.0 by weight of gel-forming agent
about 40-60 by weight of water
about 0.05-2.0 by weight of catalyst
about 0.5-2.0 by weight of coloring agent.

32. A transparent antifreeze gel, comprising
about 40-60 by weight of sorbitol
about 0.8-1.5 by weight of carrageenan gum
about 40-60 by weight of water
about 0.1-1.0 by weight of potassium chloride
about 0.5-2.0 by weight of coloring agent.
